(19) United States
(12) Patent Application Publication
(10) Pub. No.: US 2010/0036370 A1
(43) Pub. Date: Feb. 11, 2010

(54) ELECTROSURGICAL INSTRUMENT JAW STRUCTURE WITH CUTTING TIP

(76) Inventors: Al Mirel, Redwood City, CA (US); Robert J. Laird, Morrow, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/534,934

(22) Filed: Aug. 4, 2009

Related U.S. Application Data
(60) Provisional application No. 61/087,001, filed on Aug. 7, 2008.

(51) Int. Cl. A61B 18/14; A61B 18/18

(52) U.S. Cl. 606/33; 606/45; 606/52

(57) ABSTRACT

An electrosurgical instrument for scoring, welding and transecting tissue is provided. The electrosurgical instrument comprises first and second openable-closeable jaws and at least one scoring electrode extending from a distal end of at least one of the first and second openable-closeable jaws. The first and second openable-closeable jaws comprises first and second conductive tissue engaging surfaces, respectively, and first and second insulating members, respectively. The scoring electrode is coupled with at least one of the first and second insulating members. The at least one scoring electrode can be activated independently from the first and second openable-closeable jaws so that the electrosurgical instrument can independent score tissue and weld tissue.

### CROSS REFERENCE TO RELATED APPLICATIONS

The present application claims the benefit of U.S. Provisional application Ser. No. 61/087,001, filed on Aug. 7, 2008, the contents of which are incorporated in its entirety herein.

### STATEMENT REGARDING FEDERALLY SPONSORED RESEARCH OR DEVELOPMENT

Not Applicable.

### FIELD OF THE INVENTION

The present invention relates to medical devices, systems, and methods. More particularly, embodiments of the invention relate to surgical instruments, electrosurgical instruments, and surgical jaw structures for scoring, welding, sealing, and transecting tissue.

### BACKGROUND OF THE INVENTION

Various energy sources such as radiofrequency (RF) sources, ultrasound sources, and lasers have been developed to coagulate, seal or join together tissue volumes in open and laparoscopic surgeries. One preferred means of tissue-sealing relies upon the application of electrical energy to captured tissue to cause thermal effects therein for sealing purposes. Various mono-polar and bi-polar radiofrequency (RF) instruments and jaw structures have been developed for such purposes. In general, the delivery of RF energy to a captured tissue volume elevates the tissue temperature and thereby at least partially denatures proteins in the tissue. In a typical arrangement of a bi-polar radiofrequency (RF) jaw structure, each face of opposing first and second jaws comprises an electrode. RF current flows across the captured tissue between electrodes in the opposing jaws, denaturing many of the proteins in the captured tissue. Such proteins, including collagen, are denatured into a proteinaceous amalgam that intermixes and fuses together as the proteins renature. As the treated region heals over time, this biological “weld” is reabsorbed by the body's wound healing process. Another means of tissue-sealing relies upon the application of ultrasound energy to tissue captured between surgical jaws. The application of ultrasound energy to the captured tissue can likewise elevate the temperature of the captured tissue and thereby at least partially denature proteins in the tissue.

Many surgical procedures, such as enterotomies, require the scoring or dissection of soft tissue. Many commercially available jaw structures may not be ideal for both dissecting soft tissue and sealing different tissue structures during the same surgical procedure.

Currently available RF jaws that engage opposing sides of a tissue volume typically cannot cause uniform thermal effects in the tissue, whether the captured tissue is thin or substantially thick. As RF energy density in tissue increases, the tissue surface becomes desiccated and resistant to additional ohmic heating. Localized tissue desiccation and charring can occur almost instantly as tissue impedance rises, which then can result in a non-uniform seal in the tissue. Typical currently available RF jaws can cause further undesirable effects by propagating RF density laterally from the engaged tissue to cause unwanted collateral thermal damage.

For these and other reasons, there is a need for surgical instruments, electrosurgical instruments, and surgical jaw structures which avoid at least some of the aforementioned shortcomings of present devices. In particular, there is a need for electrosurgical jaw structures which can provide uniform, high strength tissue welds, reduce unwanted thermal damage, and are also adapted to score or dissect tissue as well as seal tissue structures.

### BRIEF SUMMARY OF THE INVENTION

Various embodiments of systems and methods of the invention relate to creating thermal “welds” or “fusion” within native tissue volumes. The alternative terms of tissue “welding” and tissue “fusion” may be used interchangeably herein to describe thermal treatments of a targeted tissue volume that result in a substantially uniform fused-together tissue mass, for example, in welding blood vessels that exhibit substantial burst strength immediately post-treatment. The strength of such welds is particularly useful for (i) permanently sealing blood vessels in vessel transection procedures; (ii) welding organ margins in resection procedures; (iii) welding other anatomic ducts wherein permanent closure is required; and also (iv) for performing vessel anastomosis, vessel closure or other procedures that join together anatomic structures or portions thereof.

The welding or fusion of tissue as disclosed herein is to be distinguished from “coagulation”, “hemostasis” and other similar descriptive terms that generally relate to the collapse and occlusion of blood flow within small blood vessels or vascularized tissue. For example, any surface application of thermal energy can cause coagulation or hemostasis—but does not fall into the category of “welding” as the term is used herein. Such surface coagulation does not create a weld that provides any substantial strength in the treated tissue.

At the molecular level, the phenomena of truly “welding” tissue as disclosed herein may not be fully understood. However, the authors have identified the parameters at which tissue welding can be accomplished. An effective “weld” as disclosed herein results from the thermally-induced denaturation of collagen and other protein molecules in a targeted tissue volume to create a transient liquid or gel-like proteinaceous amalgam. A selected energy density is provided in the targeted tissue to cause hydrothermal breakdown of intra- and intermolecular hydrogen crosslinks in collagen and other proteins. The denatured amalgam is maintained at a selected level of hydration—without desiccation—for a selected time interval which can be very brief. The targeted tissue volume is maintained under a selected very high level of mechanical compression to insure that the unwound strands of the denatured proteins are in close proximity to allow their intertwining and entanglement. Upon thermal relaxation, the intermixed amalgam results in protein entanglement as re-crosslinking or renaturation occurs to thereby cause a uniform fused-together mass.

Embodiments of the invention provide electrosurgical jaw structures adapted for transecting captured tissue between the jaws and for contemporaneously welding the captured tissue margins with controlled application of RF energy. The jaw structures can comprise a scoring element which can cut or score tissue independently of the tissue capturing and welding functions of the jaw structures. The jaw structures can comprise first and second opposing jaws that carry positive temperature coefficient (PTC) bodies for modulating RF energy delivery to the engaged tissue.

In a first aspect, embodiments of the invention provide an electrosurgical instrument for scoring, welding, and transecting tissue. The electrosurgical instrument comprises first and second openable-closeable jaws and at least one scoring electrode extending from a distal end of at least one of the first and second openable-closeable jaws. The first and second openable-closeable jaws comprise first and second conductive tissue engaging surfaces, respectively, and first and second insulating members, respectively. The scoring electrode is coupled with at least one of the first and second insulating members.

In many embodiments, the at least one scoring electrode comprises a wire such as a Tungsten wire. The wire may be disposed at least partially within at least one of the first and second insulating members. A distal portion of the wire may extend distally from a distal end of at least one of the first and second insulating members. The distal portion of the wire may loop back into the distal end of at least one of the first and second insulating members and terminate therein. In many embodiments, the at least one scoring electrode is electrically isolated from the first and second conductive tissue engaging surfaces.

In many embodiments, a distal portion of at least one of the first and second insulating members extends distally from a distal end of the first and second openable-closeable jaws, respectively. In many embodiments, the first and second insulating members are made of at least one of the materials selected from the group consisting of zirconia, partially stabilized zirconia, aluminum oxide, silicon nitride, alumina-chromia, hydroxyapatite, other non-conductive glass materials, other non-conductive ceramic materials, and other non-conductive glass-ceramic materials.

In many embodiments, the at least one scoring electrode is adapted to be activated to apply electrosurgical energy to score tissue. In some embodiments, the first and second openable-closeable jaws are adapted to be activated to apply electrosurgical energy to weld tissue. The at least one scoring electrode can be activated independently from the first and second openable-closeable jaws. The applied electrosurgical energy may comprise radiofrequency (RF) energy. In many embodiments, the first and second conductive tissue engaging surfaces are coupled to a radiofrequency (RF) energy source.

In many embodiments, the electrosurgical instrument further comprises a proximal handle portion and an elongate shaft. The elongate shaft has a proximal end and a distal end. The proximal end is coupled to the proximal handle portion and the distal end is coupled to the first and second openable-closeable jaws. The first and second openable-closeable jaws are adapted to be rotatable about a longitudinal axis of the elongate shaft.

In many embodiments, the electrosurgical instrument further comprises an axially-extending element actuable from a retracted position to an extended position in an axial channel defined by the first and second openable-closeable jaws. A distal portion of the axially-extending element has a cutting member adapted to transect tissue.

In many embodiments, the first and second openable-closeable jaws further comprise first and second positive temperature coefficient (PTC) bodies, respectively. In many embodiments, the first conductive tissue engaging surface has a plurality opposite of a polarity of the second conductive tissue engaging surface.

In many embodiments, the first conductive tissue engaging surface comprises a peripheral portion and a non-peripheral portion. The peripheral portion having a polarity opposite of a polarity of non-peripheral portion. In many embodiments, the second conductive tissue engaging surface comprises a peripheral portion and a non-peripheral portion. The peripheral portion having a polarity opposite of a polarity of non-peripheral portion.

In another aspect, embodiments of the invention provide a method for scoring, welding, and transecting a target tissue volume using a electrosurgical jaw. A tissue structure adjacent the target tissue volume is scored with a scoring electrode extending distally from at least one of a first openable-closeable jaw and a second openable-closeable jaw. The target tissue volume is clamped between a first tissue engaging surface and a second tissue engaging surface of the first openable-closeable jaw and second openable-closeable jaw, respectively. RF energy is delivered to the target tissue volume to weld at least a portion of the target tissue volume. The method may further comprise transecting the target tissue volume with the electrosurgical jaw.

In many embodiments, the first openable-closeable jaws and second openable-closeable jaws each comprise a first positive temperature coefficient (PTC) body portion and a second positive temperature coefficient (PTC) body portion, respectively. Delivering RF energy to the target tissue volume may comprise utilizing at least one of the first PTC body portion and the second PTC body portion to reduce tissue desiccation and charring in the target tissue volume. Delivering RF energy to the target tissue volume may comprise utilizing at least one of the first PTC body portion and the second PTC body portion to modulate a progression of ohmic heating.

### BRIEF DESCRIPTION OF THE FIGURES

The novel features of the invention are set forth with particularity in the appended claims. The invention itself, however, both as to organization and methods of operation, may best be understood by reference to the following description, taken in conjunction with the accompanying drawings in which:

FIG. 1 is a perspective view of an electrosurgical instrument according to an embodiment of the invention;

FIG. 1A is a sectional view of a translatable member of the electrosurgical instrument of FIG. 1;

FIG. 2A shows the working end of the electrosurgical instrument of FIG. 1, the working end having a scoring electrode and being in the open position;

FIG. 2B shows the working end of FIG. 2A in a closed position;

FIGS. 2C and 2D show cross-sections of the working end of FIG. 2A;

FIG. 2E is a sectional view of the lower jaw of the working end of FIG. 2A;

FIG. 2F is a block diagram of the series and parallel electrical circuit components of the working end of FIG. 2A;

FIG. 3A shows a working end of an electrosurgical instrument according to another embodiment of the invention, the working end having two scoring electrode and being in the open position;

FIG. 3B shows the working end of FIG. 3A in a closed position;

FIGS. 3C and 3D show cross-sections of the working end of FIG. 3A;

FIG. 3E show a side view of the working end of FIG. 3A;

FIG. 4A shows a working end of an electrosurgical instrument according to another embodiment of the invention, the working end having two scoring electrode and being in the open position;

FIG. 4B shows the working end of FIG. 4A in a closed position;

FIGS. 4C and 43D show cross-sections of the working end of FIG. 4A; and

FIG. 4E show a side view of the working end of FIG. 4A.

### DETAILED DESCRIPTION OF THE INVENTION

Before explaining the present invention in detail, it should be noted that the invention is not limited in its application or use to the details of construction and arrangement of parts illustrated in the accompanying drawings and description. The illustrative embodiments of the invention may be implemented or incorporated in other embodiments, variations and modifications, and may be practiced or carried out in various ways. Further, unless otherwise indicated, the terms and expressions employed herein have been chosen for the purpose of describing the illustrative embodiments of the present invention for the convenience of the reader and are not for the purpose of limiting the invention.

Further, it is understood that any one or more of the following-described embodiments, expressions of embodiments, examples, etc. can be combined with any one or more of the other following-described embodiments, expressions of embodiments, examples, etc.

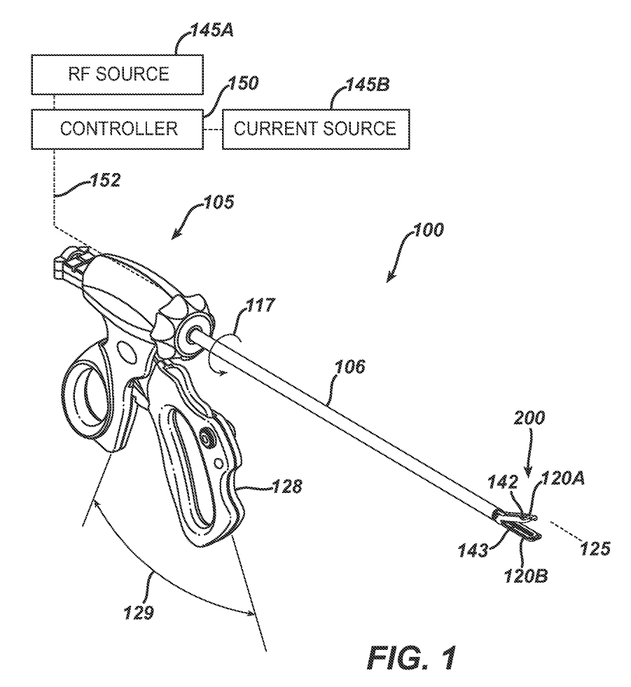

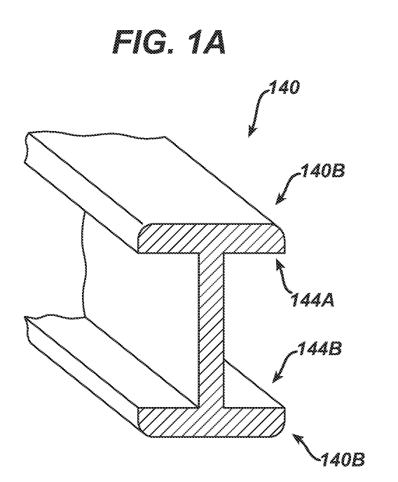

FIG. 1 shows an electrosurgical instrument 100 according to an embodiment of the invention. Electrosurgical instrument 100 comprises a proximal handle end 105, a distal working end 200 and an introducer or shaft member 106 disposed in-between. Working end 200 comprises a set of openable-closeable jaws with straight or curved jaws—an upper first jaw 120A and a lower second jaw 120B. First jaw 120A and second jaw 120B each comprise an elongate channel 142 disposed outwardly along their respective middle portions. First jaw 120A and second jaw 120B are coupled to a first electrical source or RF source 145A, a second electrical source 145B, and a controller 150 through electrical leads in cable 152. Controller 150 may be used to separately activate first electrical source 145A and second electrical source 145B.

As seen in FIG. 1, handle end 105 may comprise a lever arm 128 which may be pulled along a path 129. Lever arm 128 may be coupled to translatable, reciprocating member 140 disposed within introducer or shaft member 106. The handle can be any type of pistol-grip or other type of handle known in the art that is configured to carry actuator levers, triggers or sliders for actuating the first jaw 120A and second jaw 120B. Introducer or shaft member 106 has a cylindrical or rectangular cross-section and can comprise a thin-wall tubular sleeve that extends from handle 105. Introducer or shaft member 106 has a bore extending therethrough for carrying actuator mechanisms, for example, a translatable, reciprocating member 140, for actuating the jaws and for carrying electrical leads for delivery of electrical energy to electrosurgical components of working end 200.

Working end 200 may be adapted for capturing, welding and transecting tissue. First jaw 120A and second jaw 120B may close to thereby capture or engage tissue about an axis 125. First jaw 120A and second jaw 120B may also apply compression to the tissue. Introducer 106 along with first jaw 120A and second jaw 120B can be rotated a full 360° degrees, as shown by arrow 117, relative to handle 105 through, for example, a rotary triple contact. First jaw 120A and second jaw 120B can remain openable-closeable and operable while rotated. First jaw 120A and second jaw 120B may be coupled to a first electrical source 145A, a second electrical source 145B, and a controller 150 through electrical leads in cable 150 to function as paired bi-polar electrodes with a positive polarity (+) and a negative polarity (−).

FIG. 1A shows a portion of translatable, reciprocating member or reciprocating “I-beam” member 140. The lever arm 128 of handle 105 may be adapted to actuate translatable member 140 which also functions as a jaw-closing mechanism. For example, translatable member 140 may be urged distally as lever arm 128 is pulled proximally along path 129. The distal end of translatable member 140 comprises a flanged “I”-beam configured to slide within channels 142 in jaws 120A and 120B. Translatable member 140 slides within channels 142 to open and close first jaw 120A and second jaw 120B. The distal end of translatable member 140 comprises upper flange or “c”-shaped portion 140A and lower flange or “c”-shaped portion 140B. The flanges 140A and 140B respectively define inner cam surfaces 144A and 144B for engaging outward facing surfaces of first jaw 120A and second jaw 120B. The opening-closing of first jaw 120A and 120B can apply very high compressive forces on tissue using cam mechanisms having reciprocating “I-beam” member 140. The cam mechanism can be very well known in art. Electrosurgical working ends, jaw closing mechanisms and electrosurgical energy-delivery surfaces are described in the following U.S. patents, all of which are incorporated herein in their entirety by reference and made a part of this specification: U.S. Pat. Nos. 7,381,209; 7,354,400; 7,311,709; 7,220,951; 7,189,233; 7,186,253; 7,186,253; 7,169,147; 7,125,409; 7,112,201; 7,087,054; 7,083,619; 7,070,597; 7,041,102; 7,011,657; 6,929,644; 6,926,716; 6,913,579; 6,905,497; 6,802,843; 6,770,072; 6,656,177; 6,533,784; and 6,500,176.

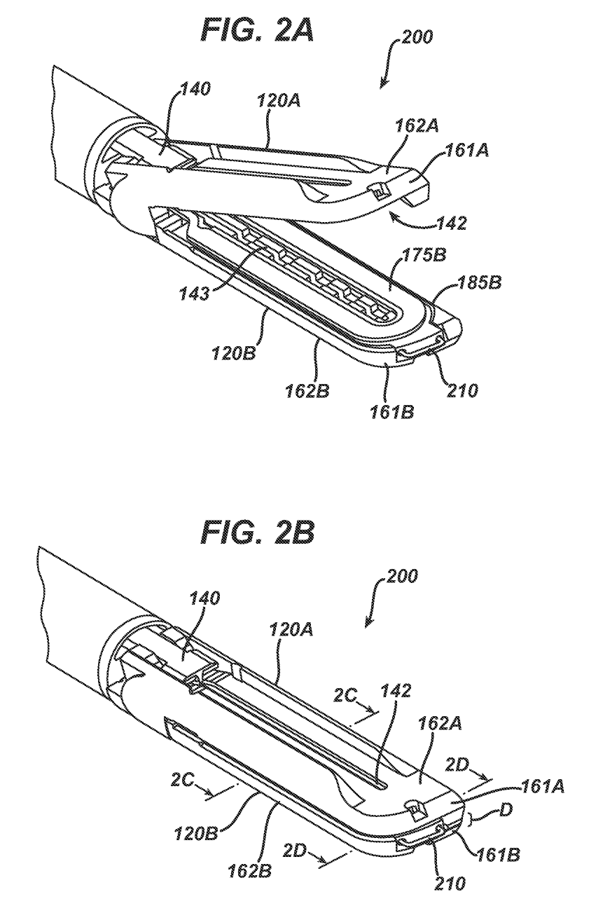

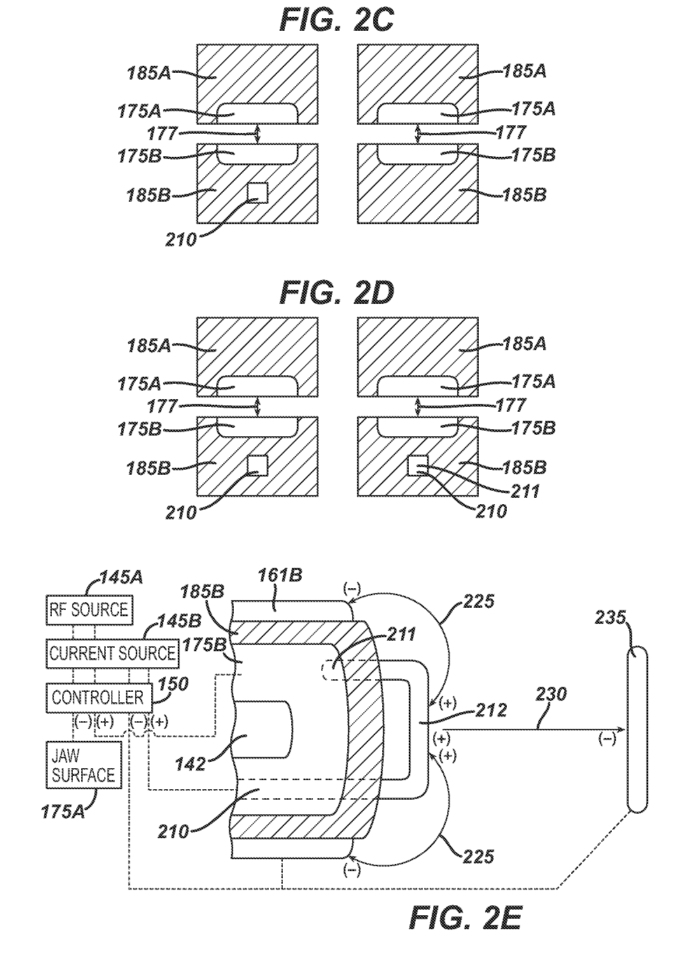

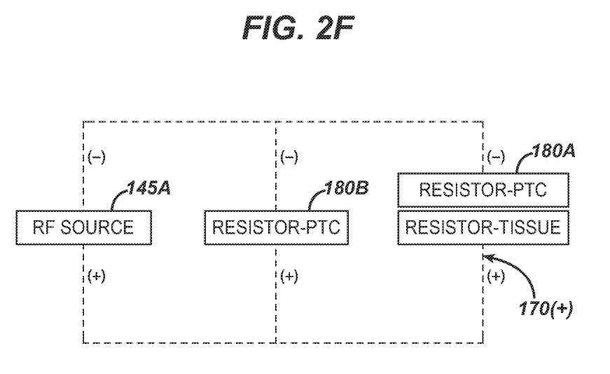

FIGS. 2A to 2E illustrate views of a working end 200. FIG. 2A shows working end 200 in an open configuration and FIG. 2B shows working end 200 in a closed configuration. FIGS. 2C and 2D show cross-sectional views of working end 200. FIG. 2E shows a cross-section of the lower jaw 120B of working end 200. FIG. 2F is a block diagram of the series and parallel electrical circuit components of the working end of FIG. 2A.

Working end 200 comprises an upper first jaw 120A and a lower second jaw 120B. First jaw 120A and second jaw 120B each have a channel 142 and tissue-gripping elements such as teeth 143 disposed on the inner portions of first jaw 120A and second jaw 120B. First jaw 120A comprises an upper first jaw body 161A with an upper first outward-facing surface 162A, an upper first energy delivery surface 175A, and an upper first intermediate member 185A. Second jaw 120B comprises a lower second jaw body 161B with a lower second outward-facing surface 162B, a lower second energy delivery surface 175B, and a lower second intermediate member 185B. First energy delivery surface 175A and second energy delivery surface 175B both extend in a “U” shape about the distal end of working end 200. First intermediate member 185A and second intermediate member 185B may comprise a thermal and/or electrical insulator, for example, zirconia, partially stabilized zirconia, aluminum oxide, silicon nitride, alumina-chromia, hydroxyapatite, other non-conductive glass materials, other non-conductive ceramic materials, and other non-conductive glass-ceramic materials. Inner cam surfaces 144A and 144B of the distal end of translatable member 140 may be adapted to slidably engage first outward-facing surface 162A and second outward-facing surface 162B of first jaw 120A and second jaw 120B, respectively. Channel 142 within first jaw 120A and second jaw 120B accommodates the movement of reciprocating member 140, which may comprise a tissue-cutting element, for example, a sharp distal edge. FIG. 2B, for example, shows the distal end of translatable member 140 advanced at least partially through channel 142. The advancement of translatable member 140 can close working end 200 from the open configuration shown by FIG. 2A. In the closed position shown by FIG. 2B, upper first jaw 120A and lower second jaw 120B define a gap or dimension D between the first energy delivery surface 175A and second energy delivery surface 175B of first jaw 120A and second jaw 120B, respectively. Dimension D equals from about 0.0005″ to about 0.005″ and preferably between about 0.001″ to about 0.002″. The edges of first energy delivery surface 175A and second energy delivery surface 175 may be rounded to prevent the dissection of tissue. Working end 200 further comprises a scoring electrode 210.

As shown in FIG. 2E, working end 200 is coupled to first electrical source 145A, second electrical source 145B, and controller 150. First energy delivery surface 175A and second energy delivery surface 175B are each coupled to first electrical source 145A and controller 150. First energy delivery surface 175A and second energy delivery surface 175B may be configured to contact tissue and delivery electrosurgical energy to engaged tissue which is adapted to seal or weld the tissue. Controller 150 can regulate the electrical energy delivered by first electrical source 145A which in turn delivers electrosurgical energy to first energy-delivery surface 175A and second energy-delivery surface 175B. Scoring electrode 210 can be coupled to second electrical source 145B and controller 150. Controller 150 can regulate the electrical energy delivered by second electrical source 145B which in turn delivers electrosurgical energy to scoring electrode 210. Scoring electrode 210 in turn delivers electrosurgical energy to cut or score tissue. The electrosurgical energy delivered by first electrical source 145A and/or second electrical source 145B may comprise radiofrequency (RF) energy.

As seen in FIGS. 2C and 2D, electrosurgical energy may be delivered through current paths 177 between first energy delivery surface 175A and second energy delivery surface 175B. Translatable member 140 may comprise an insulating layer to prevent member 140 from functioning as a conductive path for current delivery. Opposing first and second energy delivery surfaces 175A and 175B may carry variable resistive positive temperature coefficient (PTC) bodies or matrices 180A and 180B that are coupled to first electrical source 145A and controller 150 in series and parallel circuit components as shown in FIG. 2F. First energy delivery surface 175A and PTC body 180A can have a negative polarity (−) while second energy delivery surface 175B and PTC body 180B can have a positive polarity (+). PTC materials will “trip” and become resistant once a selected trip current is exceeded. First energy delivery surface 175A and second energy delivery surface 175B can carry any of the PTC matrix and electrode components disclosed in U.S. Pat. No. 6,929,644 entitled “Electrosurgical Jaw Structure for Controlled Energy Delivery” and U.S. Pat. No. 6,770,072 entitled “Electrosurgical Jaw Structure for Controlled Energy Delivery”, the disclosures of both of which are fully incorporated herein by reference. The use of PTC materials in electrosurgical instruments is also described in U.S. Pat. No. 7,112,201 entitled “Electrosurgical Jaw Structure for Controlled Energy Delivery” and U.S. Pat. No. 6,929,622 entitled “Electrosurgical Jaw Structure for Controlled Energy Delivery”, the disclosures of both of which are fully incorporated herein by reference.

As shown in FIGS. 2A, 2B and 2E, a portion of second intermediate member 185B may be exposed or may protrude distally from lower jaw body 161B. As can be seen in FIGS. 2C, 2D and 2E, scoring electrode 210 is disposed within intermediate material 185B of lower jaw 120B. Scoring electrode 210 is adapted to score or cut tissue. Scoring electrode 210 may comprise a wire, for example, a Tungsten wire. A portion of intermediate member 185B may be exposed or may extend distally from jaw body 161B. Scoring electrode 210 extends distally from this portion and loops back around so that distal end 211 of scoring electrode 210 terminates in intermediate material 185B, exposing a portion 212. Exposed portion 212 of scoring electrode 210 is adapted to deliver electrosurgical energy adapted to score or cut tissue. Because scoring electrode 210 is disposed within intermediate material 185B, scoring electrode 210 can be electrically isolated from first energy delivery surface 175A and second energy delivery surface 175B. First energy-delivery surface 175A and second energy delivery surface 175B may also be separately and independently activated, for example, by controller 150, to delivery electrical surgical energy independently from scoring electrode 210 and vice versa. Thus, working end 200 can cut and score tissue with first jaw 120A and second jaw 120B in either an open or closed position and without unwanted thermal damage from first energy-delivery surface 175A and second energy delivery surface 175B. Alternatively, scoring electrode 210 may be disposed within and protrude from a protruding portion of intermediate material 185A of upper jaw 120A.

As shown in FIG. 2E, scoring electrode 210 may operate in a monopolar mode or a bipolar mode. In the monopolar mode, electrosurgical energy may flow from exposed portion 212 of scoring electrode 210 to a return pad 235 through current path 230 to score or cut tissue. In the bipolar mode, a return path for electrosurgical energy may be provided by jaw body 161A and jaw body 161B. As shown in FIG. 2E, electrosurgical energy may flow from exposed portion 212 of scoring electrode 210 back to lower jaw body 161B through current paths 225 to score or cut tissue.

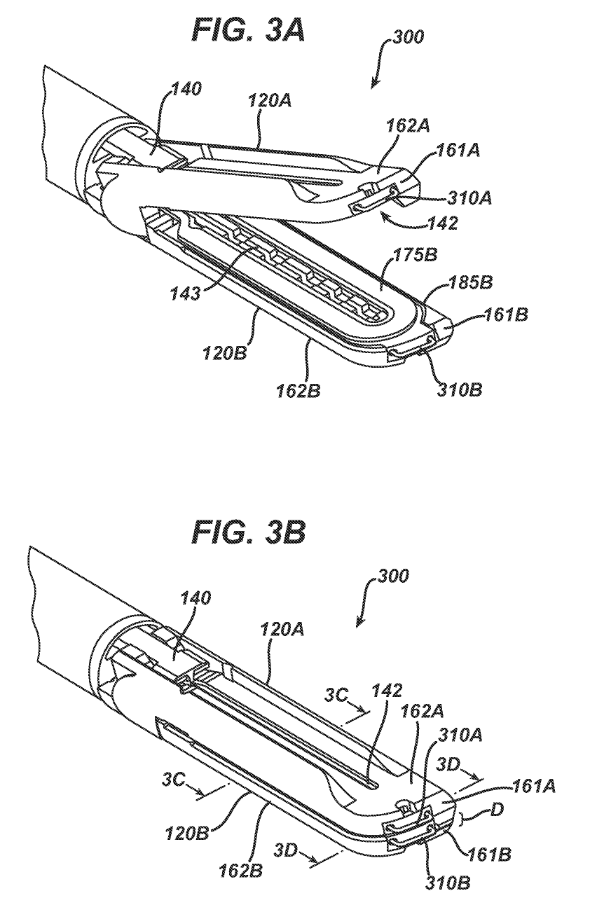

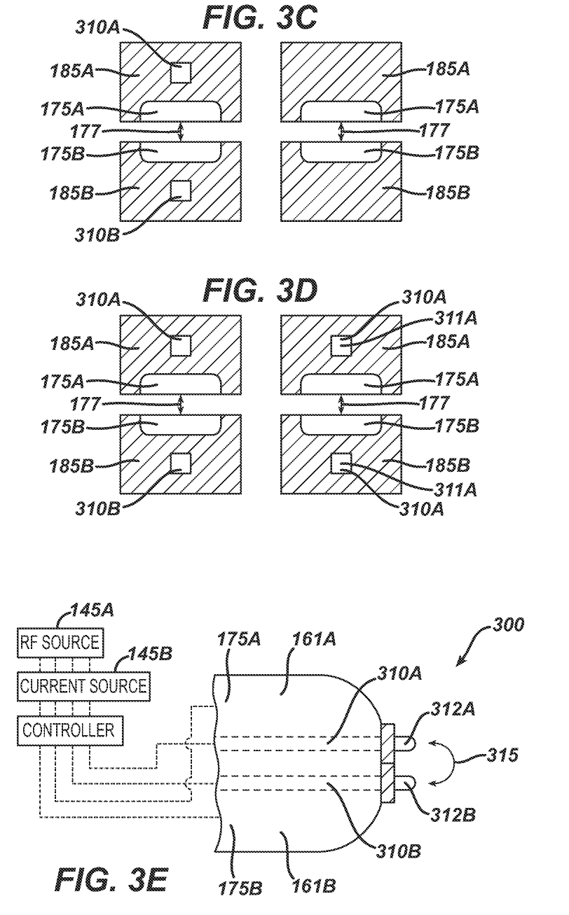

FIG. 3A to 3E illustrate views of a working end 300 which shares many features with working end 200 as described above. In another embodiment of the invention, an electrosurgical instrument comprises working end 300. Working end 300 comprises a first upper scoring electrode 310A and a second lower scoring electrode 310B. FIG. 3A shows working end 300 in an open configuration and FIG. 3B shows working end 300 in a closed configuration. FIGS. 3C and 3D show cross-section views of working end 300. FIG. 3E shows a side view of working end 300.

Scoring electrodes 310A and 310B are adapted to score or cut tissue. Scoring electrodes 310A and 310B may comprise a wire, for example, a Tungsten wire. As can be seen in FIGS. 3C, 3D and 3E, scoring electrode 310A is disposed within intermediate material 185A of upper jaw 120A and scoring electrode 310B is disposed within intermediate material 185B of lower jaw 185B. As shown in FIGS. 3A, 3B and 3E, a portion of first intermediate member 185A may be exposed or may protrude distally from upper first jaw body 161A and a portion of second intermediate member 185B may be exposed or may protrude distally from lower second jaw body 161B. First scoring electrode 310A may extend distally from the exposed portion of first intermediate member 185A and loops back around so that distal end 311A of first scoring electrode 310A terminates in first intermediate member 185A, exposing a first portion 312A. Second scoring electrode 310B may extend distally from the exposed portion of second intermediate member 185B and loops back around so that distal end 311B of second scoring electrode 310B terminates in second intermediate member 185B, exposing a second portion 312B. First exposed portion 312A and second exposed portion 312B are adapted to deliver electrosurgical energy adapted to score or cut tissue. Because first scoring electrode 310A is disposed within intermediate material 185A, which can comprise an insulator, first scoring electrode 310A is electrically isolated from first and second energy delivery surface 175A and 175B. Because second scoring electrode 310B is disposed within intermediate material 185B which can comprise an insulator, second scoring electrode 310B is electrically isolated from first and second energy delivery surfaces 175A and 175B. First and second energy delivery surfaces 175A and 175B may also be separately and independently activated to delivery electrical surgical energy from first and second scoring electrodes 310A and 310B and vice versa. Thus, working end 300 can cut and score tissue with jaws 120A and 120B either open or closed and without unwanted thermal damage from first and second energy delivery surfaces 175A and 175B.

As shown in FIG. 3E, working end 300 works in a bipolar mode. First scoring electrode 310A and 310B are each coupled to second electrical source 145B. Current can flow between exposed portion 312A of first electrode 310A and exposed portion 312B of second portion 312B to score or cut tissue.

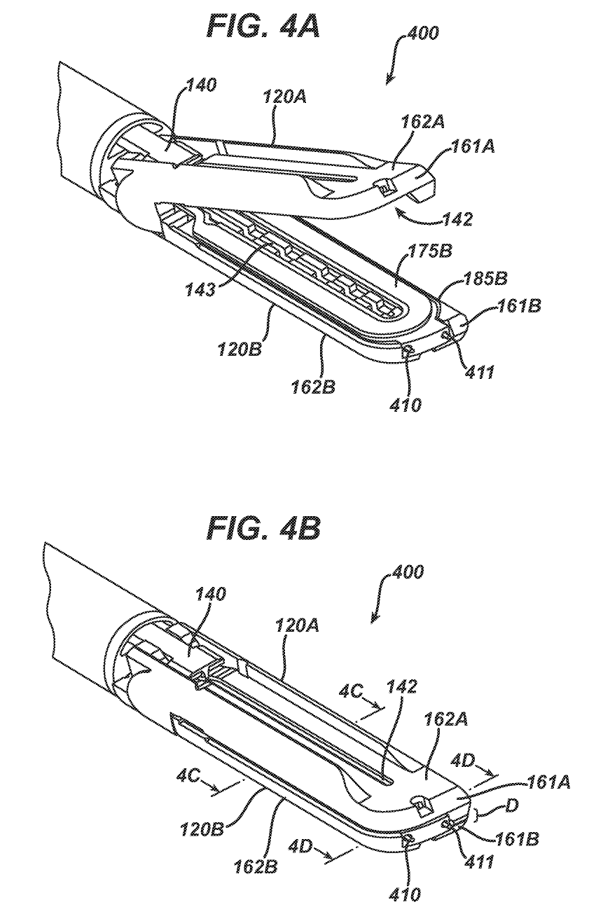

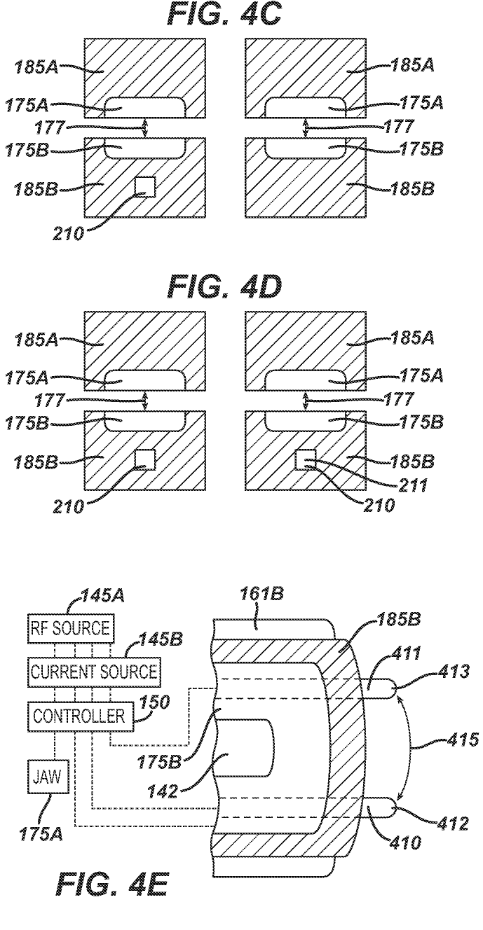

FIG. 4A to 4E illustrate views of a working end 400 which shares many features with working ends 200 and 300 as described above. In another embodiment of the invention, an electrosurgical instrument comprises working end 400 which further comprises a first scoring electrode 410 and a second scoring electrode 411. FIG. 4A shows working end 400 in an open configuration and FIG. 4B shows working end 400 in a closed configuration. FIGS. 4C and 4D show cross-section views of working end 400. FIG. 4E shows a cross-section of the lower jaw 120B of working end 400.

First scoring electrode 410 and second scoring electrode 411 are adapted to score or cut tissue. First scoring electrode 410 and second scoring electrode 411 may each comprise a wire, for example, a Tungsten wire. As can be seen in FIGS. 4C, 4D and 4E, first scoring electrode 410 and second scoring electrode 411 are disposed on opposite lateral sides of intermediate material 185B of lower jaw 185B. Alternatively, first scoring electrode 410 and second scoring electrode 411 may be disposed on opposite lateral sides of intermediate material 185A of upper jaw 185A. As shown in FIGS. 4A, 4B and 4E, a portion of second intermediate member 185B may be exposed or may protrude from lower second jaw body 161B. First scoring electrode 410 may extend from and terminate distally from the exposed portion of second intermediate member 185B. Second scoring electrode 411 may extend from and terminate distally from the exposed portion of second intermediate member 185B. Because both first scoring electrode 410 and second scoring electrode 411 are both disposed within intermediate material 185B which can comprise an insulator, first scoring electrode 410 and second scoring electrode 411 are electrically isolated from first energy-delivery surface 175A and second energy-delivery surface 175B. First energy-delivery surface 175A and second energy-delivery surface 175B may also be separately and independently activated to delivery electrical surgical energy from first scoring electrode 410 and second scoring electrode 411 and vice versa. Thus, working end 400 can cut and score tissue with first jaw 120A and second jaw 120B either open or closed and without unwanted thermal damage from first energy-delivery surface 175A and second energy delivery surface 175B.

As shown in FIG. 4E, working end 400 works in a bipolar mode. First scoring electrode 410 and second scoring electrode 411 are each coupled to second electrical source 145B. Electrosurgical energy can be delivered through current path 415 between exposed distal end 412 of first scoring electrode 410 and exposed distal end 413 of second scoring electrode 411 to score or cut tissue.

While the present invention has been illustrated by description of several embodiments, it is not the intention of the applicant to restrict or limit the spirit and scope of the appended claims to such detail. Numerous variations, changes, and substitutions will occur to those skilled in the art without departing from the scope of the invention. Moreover, the structure of each element associated with the present invention can be alternatively described as a means for providing the function performed by the element. Accordingly, it is intended that the invention be limited only by the spirit and scope of the appended claims.

We claim:

1. An electrosurgical instrument for scoring, welding, and transecting tissue, the electrosurgical instrument comprising:
first and second openable-closeable jaws, the first and second openable-closeable jaws comprising first and second conductive tissue engaging surfaces, respectively, and first and second insulating members, respectively; and
at least one scoring electrode extending from a distal end of at least one of the first and second openable-closeable jaws, the scoring electrode coupled with at least one of the first and second insulating members.

2. The electrosurgical instrument of claim 1, wherein the at least one scoring electrode comprises a wire.

3. The electrosurgical instrument of claim 2, wherein the wire comprises a Tungsten wire.

4. The electrosurgical instrument of claim 2, wherein the wire is disposed at least partially within at least one of the first and second insulating members.

5. The electrosurgical instrument of claim 2, wherein a distal portion of the wire extends distally from a distal end of at least one of the first and second insulating members.

6. The electrosurgical instrument of claim 5, wherein the distal portion of the wire loops back into the distal end of at least one of the first and second insulating members and terminates therein.

7. The electrosurgical instrument of claim 1, wherein the at least one scoring electrode is electrically isolated from the first and second conductive tissue engaging surfaces.

8. The electrosurgical instrument of claim 1, wherein a distal portion of at least one of the first and second insulating members extends distally from a distal end of the first and second openable-closeable jaws, respectively.

9. The electrosurgical instrument of claim 1, wherein the first and second insulating members are made of at least one of the materials selected from the group consisting of zirconia, partially stabilized zirconia, aluminum oxide, silicon nitride, alumina-chromia, hydroxyapatite, other non-conductive glass materials, other non-conductive ceramic materials, and other non-conductive glass-ceramic materials.

10. The electrosurgical instrument of claim 1, wherein the at least one scoring electrode is adapted to be activated to apply electrosurgical energy to score tissue.

11. The electrosurgical instrument of claim 10, wherein the first and second openable-closeable jaws are adapted to be activated to apply electrosurgical energy to weld tissue.

12. The electrosurgical instrument of claim 11, wherein the at least one scoring electrode can be activated independently from the first and second openable-closeable jaws.

13. The electrosurgical instrument of claim 11, wherein the applied electrosurgical energy comprises radiofrequency (RF) energy.

14. The electrosurgical instrument of claim 1, wherein the first and second conductive tissue engaging surfaces are coupled to a radiofrequency (RF) energy source.

15. The electrosurgical instrument of claim 1, further comprising:
a proximal handle portion; and
an elongate shaft having a proximal end and a distal end, the proximal end coupled to the proximal handle portion and the distal end coupled to the first and second openable-closeable jaws,
wherein the first and second openable-closeable jaws are adapted to be rotatable about a longitudinal axis of the elongate shaft.

16. The electrosurgical instrument of claim 1, further comprising an axially-extending element actuable from a retracted position to an extended position in an axial channel defined by the first and second openable-closeable jaws, a distal portion of the axially-extending element having a cutting member adapted to transect tissue.

17. The electrosurgical instrument of claim 1, wherein the first and second openable-closeable jaws further comprise first and second positive temperature coefficient (PTC) bodies, respectively.

18. The electrosurgical instrument of claim 1, wherein the first conductive tissue engaging surface has a plurality opposite of a polarity of the second conductive tissue engaging surface.

19. The electrosurgical instrument of claim 1, wherein the first conductive tissue engaging surface comprises a peripheral portion and a non-peripheral portion, the peripheral portion having a polarity opposite of a polarity of non-peripheral portion.

20. The electrosurgical instrument of claim 1, wherein the second conductive tissue engaging surface comprises a peripheral portion and a non-peripheral portion, the peripheral portion having a polarity opposite of a polarity of non-peripheral portion.

21. A method for scoring, welding, and transecting a target tissue volume using a electrosurgical jaw, the method comprising:
scoring a tissue structure adjacent the target tissue volume with a scoring electrode extending distally from at least one of a first openable-closeable jaw and a second openable-closeable jaw;
clamping the target tissue volume between a first tissue engaging surface and a second tissue engaging surface of the first openable-closeable jaw and second openable-closeable jaw, respectively;
delivering RF energy to the target tissue volume to weld at least a portion of the target tissue volume.

22. The method of claim 21, further comprising transecting the target tissue volume with the electrosurgical jaw.

23. The method of claim 21, wherein the first openable-closeable jaws and second openable-closeable jaws each comprise a first positive temperature coefficient (PTC) body portion and a second positive temperature coefficient (PTC) body portion, respectively.

24. The method of claim 23, wherein delivering RF energy to the target tissue volume comprises utilizing at least one of the first PTC body portion and the second PTC body portion to reduce tissue desiccation and charring in the target tissue volume.

25. The method of claim 23, wherein delivering RF energy to the target tissue volume comprises utilizing at least one of the first PTC body portion and the second PTC body portion to modulate a progression of ohmic heating.
